(19) United States
(12) Patent Application Publication
(10) Pub. No.: US 2007/0105232 A1
(43) Pub. Date: May 10, 2007

(54) Voltammetric detection of metabolites in physiological fluids

(76) Inventors: Selwayan Saini, Bedfordshire (GB); Conrad Bessant, Bedfordshire (GB); Jon Lee-Davey, Bedfordshire (GB); Michael Markus Malecha, Bedfordshire (GB)

Correspondence Address: STITES & HARBISON PLLC, 1199 NORTH FAIRFAX STREET, SUITE 900, ALEXANDRIA, VA, 22314, US

(21) Appl. No.: 10/562,229

(22) PCT Filed: Jun. 28, 2004
(86) PCT No.: PCT/GB04/02770

§ 371 Date: Oct. 27, 2006

(30) Foreign Application Priority Data
Jun. 26, 2003 (GB) 0314944.0

(51) Int. Cl. G01N 33/00

(52) U.S. Cl. 436/95

(57) ABSTRACT

For the determination of analytes in body fluids, voltammetry using a simple, non-selective, electrode sensor system has been found to work. The signal is desirably processed by a technique such as neural network analysis, which can permit plural analytes to be determined simultaneously.

### TECHNICAL FIELD

This invention relates to a method and apparatus for the electrochemical measurement or detection of one or more metabolites in body fluids such as blood, plasma or interstitial fluid. The sensor may be used for in-vitro or in-vivo applications for the determination of multiple metabolites. It may be used in solutions that contain protein and may be complex mixtures.

The concentrations of multiple metabolites in body fluids, particularly blood or interstitial fluid, are key indicators to the state of health of the body. Monitoring different metabolites is desirable when disease is present (or suspected) or when the health status of the individual is required to be assessed. For example, the level of glucose in blood provides information on the health of a diabetic patient. In addition to glucose, there are many other metabolites of clinical interest such as creatinine, cholesterol, lactate and uric acid.

There are many devices for measuring metabolites in body fluids. For diagnostics purposes, these can be broadly classified into one or more of the following groups: (i) procedures performed in a specialised laboratory such as a central blood laboratory in a hospital, (ii) techniques performed at the point of care or (iii) diagnostic devices designed for personal use. This invention has applicability to all three types of diagnostic device. Much research and development is being channelled into the advancement or generation of new devices able to improve on existing diagnostics.

This invention is concerned with diagnostic devices that are based on electrochemical sensing. Such diagnostics fall into all three categories mentioned above and a good example of a diagnostic of this ilk is the blood glucose test. Glucose sensors have been developed for laboratory, point of care and personal use. Many of the glucose sensors are based around electrochemical sensing although optical sensors are also available. In addition to glucose, it is desirable to measure other metabolites of clinical significance using devices that fall into one or more of the three groups above. Examples of other metabolites have been mentioned earlier.

### BACKGROUND ART

A variety of electrochemical diagnostic devices have been described for the determination of metabolites in body fluids. A diagnostic device in this context usually consists of a sensor component that performs the measurement and additional components that control the sensor or provide a method by which sample is delivered to the sensor. The entire assembly of components represents the diagnostic device.

Body fluids such as blood, plasma and interstitial fluid are highly complex liquid samples containing over one hundred different chemical components in addition to larger structures such as proteins and specialised cells. All of these components have the potential to interfere with the electrochemical measurement of a metabolite for diagnostic purposes. In order to obtain a useful measurement using an electrochemical sensor, devices have been designed to select for a metabolite of interest amongst all the other potential interfering compounds in the body fluid sample. This requires an approach that will select for one target metabolite and provide an output signal that is related to that target metabolite alone. Those practised in the art have used two main methods to achieve this. The first is based on the use of biological recognition; the second is based on alternative forms of selection for the metabolite of interest.

Diagnostic devices incorporating biological materials such as enzymes have proven extremely successful, especially in low cost disposable formats. The enzyme acts as a highly specific catalyst that reacts with the metabolite of interest and the reaction is monitored using an electrode system. The signal output is related to the concentration of the metabolite. One of the most developed examples of this in the diagnostics context is the blood glucose meter. This sensor uses an enzyme, glucose oxidase or glucose dehydrogenase, to react specifically with glucose molecules in blood. In order to convert the enzyme reaction into a signal that reflects the glucose concentration only, the reaction is monitored using an elaborately designed physical-chemical system that maximizes the signal due to glucose despite the presence of many potential interfering compounds. The manner by which this is achieved is the basis of different sensors. For example, many different physical-chemical means exist for converting the reaction of glucose specific enzymes to a signal for glucose concentration in blood.

Besides glucose-specific enzymes, a number of other enzymes have been employed for monitoring other metabolites in body fluids. Examples include cholesterol using the enzyme cholesterol oxidase, and lactate, using the enzyme lactate oxidase or lactate dehydrogenase. Such enzyme-metabolite pairs form the basis of different metabolite sensors with each sensor configured to maximize the signal of the target metabolite.

In recent years it has been recognized that, for many diseases, it is desirable to detect more than one metabolite, ideally at the same time. This is because the measurement of a second metabolite can indicate related health conditions that may require further treatment, so improving the overall health management of the patient. To this end, there are commercially available devices that measure blood glucose as well as other metabolites. This is achieved by employing a separate electrochemical sensor in the device that has been configured to detect the additional metabolite. Thus it is feasible to produce multiple electrochemical sensors that each provide one metabolite signal in a diagnostic device.

Whilst enzyme based electrochemical approaches have proved popular for many diagnostics, they do carry some disadvantages. One of their greatest drawbacks is the limited lifetime of the enzyme. This may be tolerable for in-vitro devices which are can employ single use disposable sensor elements (for example, electrode strips that are used with blood glucose meters) but it severely limits the possibility for implanted devices that monitor metabolites in vivo. In order to circumvent these problems, researchers have focused on the development of non-enzyme based electrochemical sensors. It is not surprising to learn that such sensors aim to provide information about a particular target metabolite, in preference to other molecules, in a manner that mimics enzyme based sensors. Therefore, non-enzyme based sensors are also configured to ensure maximum selectivity to the target analyte of interest in a sample containing many potential interferents. Many different sensor configurations have been pursued with different means to enhance the selectivity of the sensor electrode toward a particular metabolite.

We previously disclosed inventions based on single sensors for the detection of volatile compounds in gaseous mixtures [WO 02/086149] and multiple compounds in liquids [WO 00/20855]. In the latter disclosure we demonstrated the determination of individual concentrations of aliphatic compounds present in simple mixtures. Thus we showed how three aliphatic compounds could be determined in an electrolyte solution using a single electrode and chemometric techniques based on artificial neural networks. The invention was aimed at process control applications in industry where it was useful to measure aliphatic compounds in process streams. In process applications, the range of mixtures for analysis is, however, limited because there are predefined limits to achieving an optimal process.

In contrast to industrial process streams, body fluids such as blood, plasma and interstitial fluid are highly complex liquid samples containing over one hundred different chemical components in addition to larger structures such as proteins and specialised cells. Such a mixture is exceedingly complex and unique to individuals. Thus a skilled practitioner would not seriously consider using the technique disclosed in WO 00/20855 for body fluid samples such as blood given their inherent complexity. The expectation would be that a body fluid would lead to an overwhelmingly complex composite signal resulting from the enormous number of interferences that would swamp any signal resulting from the metabolites of interest.

There is another compelling reason why WO 00/20855 would be considered unsuitable for determining metabolites in body fluids. This is in anticipation of the protein bio-fouling problem. On contacting a body fluid, the electrode would become coated with a surface film of protein. The protein coat would block access of potential target metabolites to the surface. The effect is so severe that the protein layer can effectively shield the electrode from target compounds in the sample.

### DISCLOSURE OF INVENTION

Non-Specific Electrode for Multiple Metabolites in Body Fluids

Whereas enzyme and non-enzyme based electrochemical sensors are configured for the detection of one target metabolite, it is also desirable to detect multiple metabolites where this information has the potential to improve management of the disease. The prior art achieves this with additional sensor elements, each designed to detect a target metabolite. Examples include devices that have separate blood glucose and blood ketone sensors. In contrast, the present invention permits multi-metabolite sensing using a single sensor element for use in body fluids.

Despite the anticipated difficulties, we have now found that the method and apparatus of WO 00/20855 can provide sufficient analytical information in body fluids without any substantial modifications. Despite the large number of potentially interfering compounds and macromolecules in body fluids, the sensor is able to output signals that reflect the concentrations of metabolites such as glucose and uric acid. This unexpected discovery is elaborated in further detail below in a series of examples that demonstrate operation of the sensor in body fluids. The remarkable result of using the sensor in body fluids is embodied in this invention where it becomes possible to use a single, non-selective electrode for multiple metabolites in medical diagnostics applications. An example of a diagnostic application is in the monitoring of key metabolites of relevance to diabetes and its complications.

Simple parameters extracted from the observed data, such as peak height, can be calibrated to give the concentration of individual metabolites of interest. However, further chemometric processing using, for example, the multivariate regression abilities of feed forward neural networks can be used to provide more accurate measurements. Such chemometric methods also allow the quantification of more than one analyte from a single measurement, as each analyte is typically active at a particular point in the voltammetric sweep.

### BRIEF DESCRIPTION OF DRAWINGS

FIG. 1 is a schematic view of a sensor device embodying the invention.

FIG. 2 is a schematic sectional view on II-II in FIG. 1.

FIG. 3 is a graph showing a potential/time waveform suitable for use in dual pulse staircase voltammetry in embodiments of the invention.

FIG. 4 is a voltammogram showing results for different concentrations of glucose in simulated interstitial fluid with added NaOH.

FIG. 5 is a voltammogram similar to FIG. 4, without added NaOH, for electrodes with or without a Nafion layer.

FIG. 6 is a voltammogram showing results for simulated interstitial fluid alone (“ISF”) or containing glucose (“G”), uric acid (“U”) or both glucose and uric acid (“G+U”).

FIG. 7 is a diagram for explaining the use of neural network analysis for treating the data.

### MODES FOR CARRYING OUT THE INVENTION

The invention may be carried out using one or more electrochemical cells that contact the body fluid sample. Each electrochemical cell contains one working electrode that may perform simultaneous measurement of plural metabolites. Each electrochemical cell also contain one reference electrode and one auxiliary or counter electrode. The working and counter electrodes used in the electrochemical cell can be of any shape, size, material and configuration. In a preferred embodiment, working and counter electrodes are made of a noble metal electrode material such as a platinum or gold. The reference electrode can be silver/silver chloride or other suitable reference material. Alternatively, a quasi-reference electrode could be used made from a metal or carbon material. Each electrode is electrically connected to an electronic potentiostat device that controls the voltage difference between the working electrode and the reference electrode and measures the current resulting from redox reactions at the working electrode.

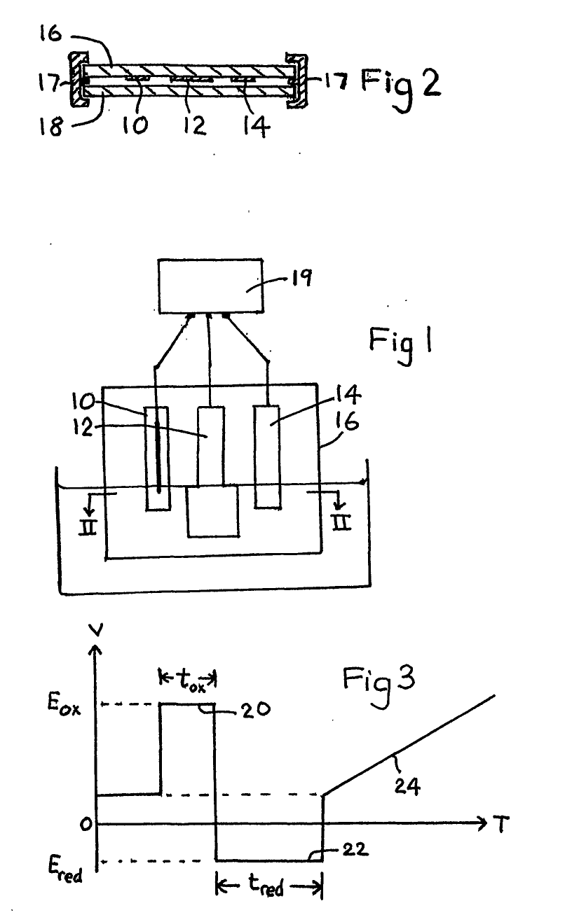

In a preferred embodiment, the three electrodes are part of an electrochemical cell which is delimited by a physical support onto which the electrodes are formed. A variety of cell configurations can be used for the detector. A preferred design is shown schematically in FIGS. 1 and 2. In this embodiment, the three electrodes (reference electrode 10, working electrode 12 and counter-electrode 14) are of a planar configuration and have been formed onto a suitable substrate material 16 such as a glass, ceramic or plastic substrate. A variety of methods may be used to form the electrodes including thin film techniques such as vapour deposition of the working and counter electrodes using materials such as platinum or gold. The reference electrode can be formed from thick film techniques based on for example, the screen printing of conducting pastes. This method equally applies to the formation of the working and counter electrodes.

The electrodes are connected to a potentiostat device 19 which may incorporate data processing means, or be connectable to an external computer.

The method of introducing a body fluid onto the electrodes, enabling electrochemical measurement can be through a number of means. In one embodiment, capillary action is used to fill the electrochemical cell. Capillary action is a physical effect caused by the interactions of a liquid with the walls of a vessel. The capillary effect is a function of the ability of the liquid to wet a particular vessel material, most usually glass. In the preferred embodiment, the three electrodes 10,12,14 are formed in a planar design onto a planar glass substrate 16. An additional glass cover 18 (FIG. 2) is provided above the electrodes so that the distance between the two glass walls is minimal to allow capillary action of a body fluid to operate so that it fills the formed electrochemical cell. The walls 16, 18 may be spaced and sealed by side seals 17. Other electrode and cell configurations using capillary action of the body fluid could also be used. In yet a further embodiment, a method of active transport may be used such as a sample pump that delivers sample to the electrochemical cell.

In some embodiments, reagents may be present in the electrochemical cell that facilitate the measurement of multiple metabolites in the body fluid. These additional materials function to enhance the electrochemical response of the target metabolites. Examples include NaOH or other material capable of generating an alkaline environment around the electrodes. The formation of OH₋ ions that lead to alkaline conditions in the vicinity of the working electrode may be formed chemically or electrochemically. Other materials that cause an acidic environment may also be used. Other materials may also be present as part of the electrochemical cell such as membranes that coat the electrode or have been placed elsewhere in the cell. Examples of membrane materials are Nafion, cellulose acetate, polyurethane, Kel F and polyvinyl chloride.

Several voltage-time waveforms can be used to incite electrochemical redox reactions of multiple metabolites in a body fluid. An example is the waveform shown in FIG. 3. This was previously described in WO 00/20855. This consists of two cleaning pulses (oxidising 20 and reducing 22), which clear the electrode of any electrochemical breakdown products from previous measurements, followed by a voltammetric sweep 24 (generally linear) during which current measurement takes place.

In an example described in WO 00/20855. each DPSV scan consisted of a 3 s 0.7V pulse, to remove adsorbed fouling agents and form platinum oxide on the electrode surface, and a 2 s −0.9V pulse to regenerate the surface by removing the oxide layer, followed by a scan from −0.9V to 0.2V in steps of 10 mV at a rate of 0.5V.s⁻¹. The current was recorded at the end of each potential step during the scan. Such parameters can be tuned in order to enhance the detection of metabolites.

Other variations of voltage-time waveforms could also be used to incite electrochemical redox reactions such as square wave voltammetry, differential pulse voltammetry, normal pulse voltammetry and cyclic voltammetry. In a different embodiment, a dual cleaning pulse is omitted prior to the linear voltage sweep or other scanning voltammetric method.

Simple parameters extracted from the observed data, such as peak height, can be calibrated to give the concentration of individual metabolites of interest. However, further chemometric processing using, for example, the multivariate regression abilities of feed forward neural networks can be used to provide more accurate measurements.

Sensor devices utilising this invention may be operated as in-vitro or in-vivo and could be used in conjunction with a variety of body fluids such as blood, plasma, interstitial fluid, urine, sputum or any other body fluid sample.

### EXAMPLE 1

Glucose Detection in Interstitial Fluid

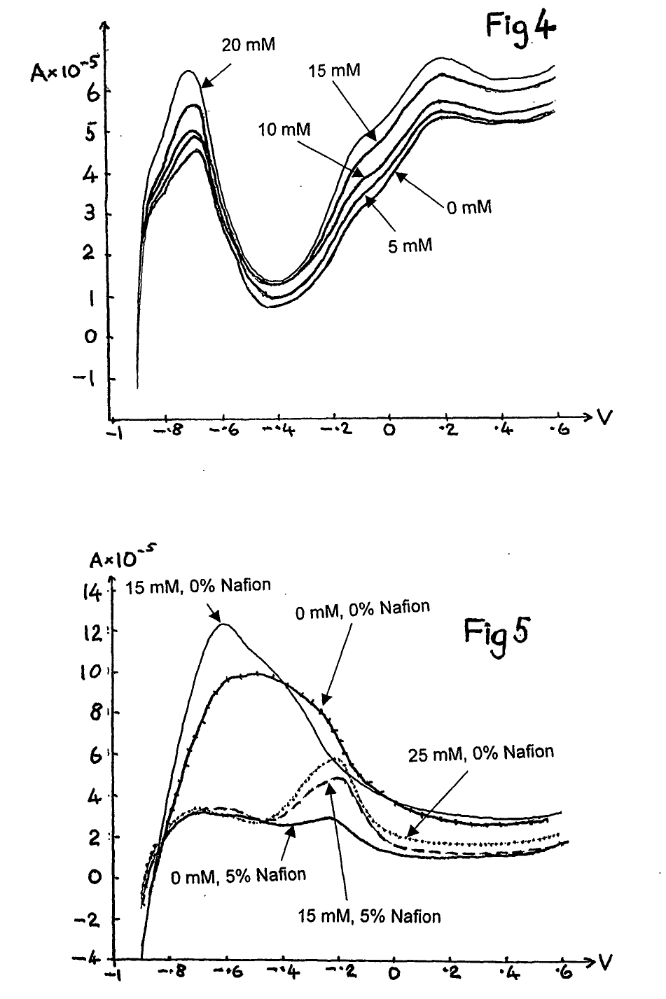

In this example, the body fluid was interstitial fluid (ISF) which was closely approximated using human blood plasma (obtained from the centrifugation of whole blood) then diluted to 33% v/v with phosphate buffer saline solution. Several mixtures of ISF were prepared containing different concentrations of glucose (0, 5, 10, 15 and 20 mM) and 0.1M NaOH electrolyte. On taking electrode measurements in each mixture, resulting voltammmograms showed distinctive glucose peaks as a function of glucose concentration, as shown in FIG. 4. Improvements to the signal could be obtained by optimizing the experimental parameters such as scan rate and NaOH ionic strength. The observed glucose signal provided a surprisingly large response across a wide concentration range despite the presence of various potential interfering compounds inherent to ISF.

The glucose signal became attenuated when NaOH was omitted from ISF or when a predominantly plasma rich sample was used (80% v/v plasma). It was found that the glucose signal could be improved by changing the measurement variables such as scan rate or coating the electrode with a thin membrane, Nafion, to exclude the largest macromolecules but still allow passage of glucose and other molecules through the membrane.

FIG. 5 shows a typical response for glucose in ISF with and without the Nafion and in the absence of NaOH. Nafion was cast from a commercial solution preparation.

Metabolite detection in higher plasma volumes became more difficult owing to the overall higher concentration and numbers of interfering compounds contributing to a higher level of background signal noise. When metabolites such as glucose are present at a sufficient concentration, they elicit a signal above the background noise which can be used for analytical purposes. Optimisation of the measurement parameters and employing membranes overcomes the greater background noise levels in these body fluids.

### EXAMPLE 2

Glucose and Uric Acid Detection in Interstitial Fluid

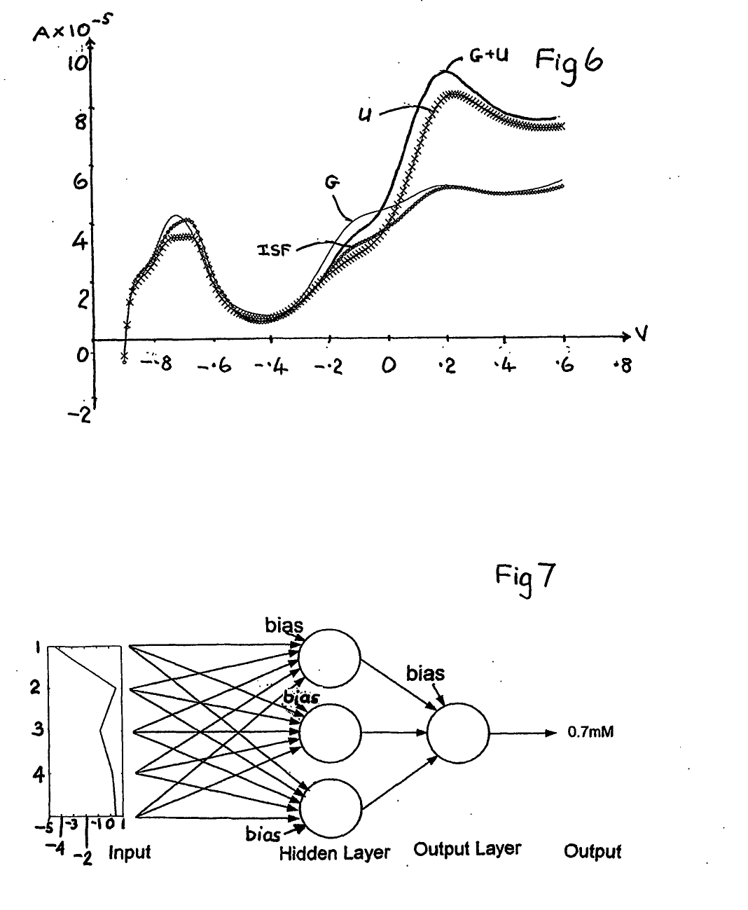

This example demonstrates the measurement of two different metabolites in ISF. As mentioned earlier, there is increasing interest in measuring more than one metabolite for diagnostic purposes. For example, in diabetes, the levels of uric acid may act as a strong predictor for stroke and for coronary heart disease. In this example, the metabolites are measured simultaneously using a single electrode sensor in a body fluid. This is in contrast to the prior art where different sensors are used for each metabolite. In order to demonstrate this aspect of the invention, glucose and uric acid were mixed into an ISF sample prepared as described in example 1. FIG. 6 shows the results obtained for the individual and simultaneous detection of glucose and uric acid. Uric acid and glucose both display individual and combined current voltage features suggesting there is sufficient information in the measurement to allow subsequent multivariate calibration, e.g. as described in WO 00/20855 or WO 02/086149 e.g. using artificial neural networks or other known techniques of multivariate statistical analysis. FIG. 7 illustrates the use of neural network calibration of sensor data. In this case, the number of inputs to the network is optimised by reducing the number of points in the acquired voltammogram using linear algebra.

1. A method for determining one or more analytes in a body fluid comprising immersing in said fluid a set of electrodes comprising a working electrode, a reference electrode and a counter electrode; applying a varying potential to the working electrode and measuring the electrochemical outcome, thereby providing an output signal related to the composition of the fluid.

2. A method according to claim 1 wherein said fluid is selected from interstitial fluid, whole blood, plasma, urine and saliva.

3. A method according to claim 2 wherein said fluid is interstitial fluid.

4. A method according to any preceding claim wherein the application of the varying potential is preceded by the application of one or more electrode cleaning pulses.

5. A method according to any preceding claim wherein the output signal is analysed to provide data about the concentration of one or more analytes.

6. A method according to claim 5 wherein the output signal is analysed to provide data about the concentration of plural analytes.

7. A method according to claim 5 or claim 6 wherein the analysis employs a multivariate calibration technique.

8. A method according to any preceding claim wherein fluid is made more alkaline or acidic prior to determination.

9. A method according to any preceding claim wherein said electrodes are film electrodes provided on a substrate.

10. A method according to any preceding claim wherein the electrodes are provided within a capillary element which is partly immersed in the fluid whereupon fluid rises into the element by capillary action to contact the electrodes.

11. A method according to any preceding claim wherein the electrodes are coated with a semipermeable film that permits passage of the analyte(s) but not proteins.

12. Apparatus for carrying out the method of any preceding claim, comprising an electrode assembly and capillary means for conveying fluid for analysis to the electrodes.

13. Apparatus according to claim 12 wherein the capillary means contain means for rendering the fluid more alkaline or acidic.
